(19) United States
(12) Patent Application Publication
(10) Pub. No.: US 2014/0275878 A1
(43) Pub. Date: Sep. 18, 2014

(54) METHODS AND SYSTEMS FOR EQUALIZING PHYSIOLOGICAL SIGNALS

(71) Applicant: COVIDIEN LP, Mansfield, MA (US)

(72) Inventor: Daniel Lisogurski, Boulder, CO (US)

(73) Assignee: Covidien LP, Mansfield, MA (US)

(21) Appl. No.: 13/842,571

(22) Filed: Mar. 15, 2013

(51) Int. Cl. A61B 5/00; A61B 5/024; A61B 5/1455

(52) U.S. Cl. CPC A61B 5/0082; A61B 5/14551; A61B 5/02416
USPC 600/323; 600/476; 600/479; 600/480

(57) ABSTRACT

A physiological monitoring system may determine an equalized physiological signal. The system may receive a light signal that includes undesired signal features associated with a channel response. The system may equalize the light signal to mitigate the undesired signal features. The equalization may be performed on an analog signal or a digital signal. The equalization may include, for example, applying an inverse response of the channel response, such that undesired signal features are mitigated while signal features associated with a subject are retained. The equalization may be implemented with, for example, a finite impulse response filter.

The present disclosure relates to operating a physiological monitor, and more particularly relates to equalizing signals of a physiological monitor such as a pulse oximeter or other medical device.

### SUMMARY

Methods and systems are provided for equalizing physiological signals of a physiological monitor such as a pulse oximeter. In some embodiments, a light signal is received. The light signal may include features corresponding to a channel response and features corresponding to a subject. An equalizer may be used to mitigate undesired features corresponding to the channel response. For example, the channel may include a high pass filter to attenuate ambient light, which may create droop and/or phase shifts in a square wave passing through the channel. The received light signal may be equalized to mitigate undesired features, for example by applying the inverse of the channel response using a finite impulse response (FIR) filter. A physiological parameter, for example blood oxygen saturation, may be determined based on the equalized signal. Equalizer coefficients may be determined based on training signals and/or channel modeling.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 is a block diagram of an illustrative physiological monitoring system in accordance with some embodiments of the present disclosure;

FIG. 2A shows an illustrative plot of a light drive signal in accordance with some embodiments of the present disclosure;

FIG. 2B shows an illustrative plot of a detector signal in accordance with some embodiments of the present disclosure;

FIG. 3 is a perspective view of an embodiment of a physiological monitoring system in accordance with some embodiments of the present disclosure;

FIG. 4 is a block diagram of an optical monitoring system in accordance with some embodiments of the present disclosure;

FIG. 5 shows an illustrative signal chain that equalizes a signal in accordance with some embodiments of the present disclosure;

FIG. 6 shows an illustrative signal chain including a test subject in accordance with some embodiments of the present disclosure;

FIG. 7 shows illustrative signals including time domain equalization in accordance with some embodiments of the present disclosure; and

FIG. 8 shows an illustrative flow diagram including steps for determining a physiological parameter in accordance with some embodiments of the present disclosure.

### DETAILED DESCRIPTION OF THE FIGURES

The present disclosure is directed towards channel equalization in, for example, a physiological monitor. In some embodiments, a system may generate signals that are transmitted to a test subject and receive signals from a test subject, where the received signals include at least a portion of the emitted signal as well as physiological information. For example, the emitted signal may be a modulated light signal with a specific pulse shape or pattern. The emitted signal may travel through a test subject's tissue and be received by a detector, for example a photo-detector which converts received light in to electrical signal. In some embodiments, undesirable signal features may be introduced to the received signal by the signal channel. The signal channel may include, for example, any suitable system elements that the signal passes through other than the test subject. Thus, the received signal may include changes corresponding to undesired channel effects and to physiological information.

In some embodiments, distortion, noise, and other signal features are defined in relation to a channel. The channel may include any suitable elements in the path between the transmitter and receiver or digitized signal. As used herein, the channel is not considered to include the test subject, such that channel effects can be mitigated by, for example, an equalizer. In some embodiments, the channel includes all elements other than the test subject. The channel may include, for example, electrical components such as digital to analog converters, current sources, LEDs, photo-detectors, trans-impedance amplifiers, passive and active filters, any other suitable components, or any combination thereof.

In the case of a pulse oximeter, or other devices utilizing a light signal, the channel may include any suitable elements of light drive signal generation, light drive signal transmission, light drive signal amplification, light drive signal filtering, light signal generation, light signal transmission, light signal filtering, light signal detection, detected light signal transmission, detected light signal amplification, detected light signal filtering, any other suitable processing, suitable filtering, suitable amplification, or any combination thereof. In the example illustrated in FIG. 4 below, the channel includes transmission of a light drive signal, a light emitter, a light detector, and light signal receiving circuitry.

In some embodiments where the signal interacts with a test subject, the signal may pass through a first portion of the channel, followed by the test subject, followed by a second portion of the channel. Thus, it will be understood that elements of the channel need not be contiguous. It will be understood that any suitable elements may be included or excluded from the channel as it is considered herein. For example, the channel may include transmission and generation of a light signal, but need not include generation of the light drive signal. In another example, the channel may include the equalizer, such that the equalizer corrects for any unwanted effects it may itself introduce.

In some embodiments, a channel response may include undesired signal features caused by, for example, channel components. In some embodiments, components of the channel are non-ideal, in that they may add noise, gain, phase shifts, droop, overshoot, undershoot, slow rise time, slow fall time, frequency clipping, ringing, waveform shaping, other undesirable elements, or any combination thereof. In some embodiments, an equalizer is used to remove these undesired elements from the received signal. In an example where a square light pulse passes through a channel, the low pass effect of the channel may slow the rise time of the pulse. For example, a high pass filter used to attenuate ambient light may cause undesired droop and/or phase shifts in a square wave signal. In another example, the response rate of a photodetector may cause undershoot in detection of a square light pulse. In another example, the frequency response of an analog-to-digital converter that receives a square wave may cause signal errors. In another example, the received light pulse may not immediately return to zero when a light drive signal is shut off. Thus, the resulting waveform in any or all of these examples may be filtered, attenuated, phase shifted and spread out in time due to the response of the channel.

In some embodiments, phase shifting may occur when square light pulses pass through the channel. A square pulse may be represented by a wide frequency spectrum of sinusoids. The channel may impose varying amounts of droop, rise and fall time, and other distortions on each respective frequency, which may result in a spreading of a square pulse.

In some embodiments, undesired feature elements may include intersymbol interference. Intersymbol interference occurs when, for example, adjacent information in a signal overlaps due to signal spread and distortion, and results in signal errors. For example, adjacent digital bits may spread due to RF interference in a communication channel, and this may result in bit errors. In another example, analog filters and patient cable effects may spread a light drive waveform out in the time domain. In an example, driving a light source such as an LED with a light drive pulse results in receiving at the detector the impulse response of the channel (e.g., filtered, attenuated and spread in time) rather than receiving an exact replicate of the transmitted impulse function. As a result, for example, during an “off” period in a light drive signal, the current provided to an LED may not immediately reach 0 mA (or any suitable turn-off level), or may decay more slowly than desired. In another example, intersymbol interference due to bandwidth limitations in a channel introduce a filter or filter effect, multipath propagation, other suitable signal channel effects, or any combination thereof. In some embodiments, equalizing a channel response may include removing intersymbol interference.

In some embodiments, an equalizer may be used to mitigate undesired signal features corresponding to a channel response. Mitigation may include attenuating, correcting, reducing, counteracting, or any other suitable change corresponding to altering the presence of some or all of one or more signal features, such as features in the channel response. In some embodiments, mitigating channel effects may enhance determination of physiological parameters.

An equalizer may be a time domain equalizer, a frequency domain equalizer, any other suitable equalizer in any suitable domain, or any combination thereof. In some embodiments, a time domain equalizer may be implemented as a finite impulse response (FIR) filter. An FIR filter may apply filter coefficients to finite section of time domain data in order to mitigate the effects of the channel response on a received signal. In some embodiments, equalization may be implemented using digital techniques, analog techniques, or any combination thereof.

In another example of time domain filtering that may be used additionally or alternatively to an FIR filter, a time domain equalizer may receive a signal and partition it into samples, such that each sample is a section of time. A particular amount of gain may be applied to each sample. A gain may be an amplification factor that is associated with that particular sample. For example, in a periodic signal, the first sample may correspond to the first 10 milliseconds, and the signal amplitude of that sample may be multiplied by a gain factor of, for example, 2.

Gains applied by any suitable equalizer, as described above, may but need not be a linear gain. For example, gains may be non-linear, such that low amplitude signals are amplified more than high amplitude signals. In another example, gain may be linear, such that all amplitudes are amplified by the same amount. In some embodiments, gain may include time information, such that a gain varies in time based on, for example, prior signal information. In some embodiments, the application of gains may include finite impulse response and/or infinite impulse response filtering techniques. It will also be understood that amplification may include a decrease in amplitude.

In some embodiments, an equalizer may be configured based on calibration, modeling, iterative refinements, any other suitable configuration steps, or any combination thereof. In some embodiments, undesirable channel effects change relatively more slowly than physiological parameters. Thus, the channel effects may be determined and substantially removed from the received signal in order to recover physiological information. For example, the dominant unwanted channel effects may come from, for example, known electronic and optical components. In some embodiments, parameters contributing to channel effects may vary depending on the particular system being used, and may include, for example, variations in temperature, component tolerances, and different patient cable types. In some embodiments, these parameters may be estimated and incorporated into the model. In an example, the effects of temperature on channel effects may be modeled and incorporated into an equalizer by using an ambient temperature probe. In another example, the channel effects of a particular probe may be considered in equalization by the system recognizing the particular probe and retrieving predetermined stored parameters. Removable components such probes, sensors or patient cables can be identified by the system, for example by sending known signals in to different pins, by a resistor ID, or by an embedded non-volatile memory.

In some embodiments, the equalizer may be configured based on an emitter and detector that bypass a test subject. The system may include at least a first light emitter directed towards a test subject while in operation, and a second emitter that bypasses the test subject and is received directly by the same or a different detector. The bypassed signal may be used to determine the channel response independent of the subject, and thus may be used to determine equalization parameters. The equalization parameters may be used to remove the channel response from the signal that is partially attenuated by the test subject. In some embodiment, a received signal is collected over a long duration relative to physiological changes, and is used to determine a channel response and/or to refine equalization parameters. Over a relatively long time period, the test subject response may average out to zero or approximately zero.

In some embodiments, the physiological signals from one or more sensors may be used by a physiological monitor to determine one or more physiological parameters of the subject. The sensors may include a light source for emitting light which may pass through perfused tissue of the subject. After the light has passed through the tissue of the subject, it may be received by a detector. The detector may provide a signal proportional to the intensity of the received light. In some embodiments, the physiological signal provided by the detector may be representative of physiological information about the blood of the subject since light of different wavelengths passing through tissue may be differentially absorbed depending on, for example, oxygen saturation of the blood. The physiological monitor may analyze the physiological signal to determine one or more physiological parameters such as pulse rate, respiration rate, respiration effort, blood oxygen saturation, blood pressure, any other suitable physiological parameter, or any combination thereof. Exemplary embodiments of determining respiration rate are disclosed in Addison et al. U.S. Patent Publication No. 2011/0071406, published Mar. 24, 2011, which is hereby incorporated by reference herein in its entirety. Exemplary embodiments of determining respiration effort are disclosed in Addison et al. U.S. Patent Publication No. 2011/0004081, published Jan. 6, 2011, which is hereby incorporated by reference herein in its entirety. Exemplary embodiments of determining blood pressure are disclosed in Addison et al. U.S. Patent Publication No. 2011/0028854, published Feb. 3, 2011, which is hereby incorporated by reference herein in its entirety. Pulse oximetry may be implemented using a photoplethysmograph. Pulse oximeters and other photoplethysmograph devices may also be used to determine other physiological parameter and information as disclosed in: J. Allen, “Photoplethysmography and its application in clinical physiological measurement,” Physiol. Meas., vol. 28, pp. R1-R39, March 2007; W. B. Murray and P. A. Foster, “The peripheral pulse wave: information overlooked,” J. Clin. Monit., vol. 12, pp. 365-377, September 1996; and K. H. Shelley, “Photoplethysmography: beyond the calculation of arterial oxygen saturation and heart rate,” Anesth. Analg., vol. 105, pp. S31-S36, December 2007; all of which are incorporated by reference herein in their entireties.

It will be understood that while many of the examples described herein refer to square waves, the system may use any suitable wave. For example, the system may use a sinusoidal light drive pulse to generate sinusoidal waves. In some embodiments, sinusoidal waves may not reach zero during what would, in a square light drive, be considered an “off” period.

The foregoing techniques may be implemented in an oximeter. An oximeter is a medical device that may determine the oxygen saturation of an analyzed tissue. One common type of oximeter is a pulse oximeter, which may non-invasively measure the oxygen saturation of a patient's blood (as opposed to measuring oxygen saturation directly by analyzing a blood sample taken from the patient). Pulse oximeters may be included in patient monitoring systems that measure and display various blood flow characteristics including, but not limited to, the oxygen saturation of hemoglobin in arterial blood. Such patient monitoring systems may also measure and display additional physiological parameters, such as a patient's pulse rate and blood pressure.

An oximeter may include a light sensor that is placed at a site on a patient, typically a fingertip, toe, forehead or earlobe, or in the case of a neonate, across a foot or hand. The oximeter may use a light source to pass light through blood perfused tissue and photoelectrically sense the absorption of the light in the tissue. In addition, locations which are not typically understood to be optimal for pulse oximetry serve as suitable sensor locations for the blood pressure monitoring processes described herein, including any location on the body that has a strong pulsatile arterial flow. For example, additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, and around or in front of the ear. Suitable sensors for these locations may include sensors for sensing absorbed light based on detecting reflected light. In all suitable locations, for example, the oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some embodiments, the photonic signal interacting with the tissue is selected to be of one or more wavelengths that are attenuated by the blood in an amount representative of the blood constituent concentration. Red and infrared (IR) wavelengths may be used because it has been observed that highly oxygenated blood will absorb relatively less red light and more IR light than blood with a lower oxygen saturation. By comparing the intensities of two wavelengths at different points in the pulse cycle, it is possible to estimate the blood oxygen saturation of hemoglobin in arterial blood.

The system may process data to determine physiological parameters using techniques well known in the art. For example, the system may determine blood oxygen saturation using two wavelengths of light and a ratio-of-ratios calculation. The system also may identify pulses and determine pulse amplitude, respiration, blood pressure, other suitable parameters, or any combination thereof, using any suitable calculation techniques. In some embodiments, the system may use information from external sources (e.g., tabulated data, secondary sensor devices) to determine physiological parameters.

In some embodiments, a light drive modulation may be used. For example, a first light source may be turned on for a first drive pulse, followed by an off period, followed by a second light source for a second drive pulse, followed by an off period. The first and second drive pulses may be used to determine physiological parameters. The off periods may be used to determine detected ambient signal levels, reduce overlap of the light drive pulses, allow time for light sources to stabilize, allow time for detected light signals to stabilize or settle, reduce heating effects, reduce power consumption, for any other suitable reason, or any combination thereof.

It will be understood that the light signal equalization techniques described herein are not limited to pulse oximeters and may be applied to any suitable medical and non-medical devices. For example, the system may include probes for regional saturation (rSO2), respiration rate, respiration effort, continuous non-invasive blood pressure, saturation pattern detection, fluid responsiveness, cardiac output, any other suitable clinical parameter, or any combination thereof.

The following description and accompanying FIGS. 1-8 provide additional details and features of some embodiments of the present disclosure.

FIG. 1 is a block diagram of an illustrative physiological monitoring system 100 in accordance with some embodiments of the present disclosure. System 100 may include a sensor 102 and a monitor 104 for generating and processing physiological signals of a subject. In some embodiments, sensor 102 and monitor 104 may be part of an oximeter. In some embodiments, all or some of sensor 102, monitor 104, or both, may be referred to collectively as processing equipment.

Sensor 102 of physiological monitoring system 100 may include light source 130 and detector 140. Light source 130 may be configured to emit photonic signals having one or more wavelengths of light (e.g. red and IR) into a subject's tissue. For example, light source 130 may include a red light emitting light source and an IR light emitting light source, e.g. red and IR light emitting diodes (LEDs), for emitting light into the tissue of a subject to generate physiological signals. In one embodiment, the red wavelength may be between about 600 nm and about 700 nm, and the IR wavelength may be between about 800 nm and about 1000 nm. It will be understood that light source 130 may include any number of light sources with any suitable characteristics. In embodiments where an array of sensors is used in place of single sensor 102, each sensor may be configured to emit a single wavelength. For example, a first sensor may emit only a red light while a second may emit only an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 140 may be chosen to be specifically sensitive to the chosen targeted energy spectrum of light source 130.

In some embodiments, detector 140 may be configured to detect the intensity of light at the red and IR wavelengths. In some embodiments, an array of sensors may be used and each sensor in the array may be configured to detect an intensity of a single wavelength. In operation, light may enter detector 140 after passing through the subject's tissue. Detector 140 may convert the intensity of the received light into an electrical signal. The light intensity may be directly related to the absorbance and/or reflectance of light in the tissue. That is, when more light at a certain wavelength is absorbed or reflected, less light of that wavelength is received from the tissue by detector 140. After converting the received light to an electrical signal, detector 140 may send the detection signal to monitor 104, where the detection signal may be processed and physiological parameters may be determined (e.g., based on the absorption of the red and IR wavelengths in the subject's tissue). In some embodiments, the detection signal may be preprocessed by sensor 102 before being transmitted to monitor 104.

In the embodiment shown, monitor 104 includes control circuitry 110, light drive circuitry 120, front end processing circuitry 150, back end processing circuitry 170, user interface 180, and communication interface 190. Monitor 104 may be communicatively coupled to sensor 102.

Control circuitry 110 may be coupled to light drive circuitry 120, front end processing circuitry 150, and back end processing circuitry 170, and may be configured to control the operation of these components. In some embodiments, control circuitry 110 may be configured to provide timing control signals to coordinate their operation. For example, light drive circuitry 120 may generate a light drive signal, which may be used to turn on and off the light source 130, based on the timing control signals. The front end processing circuitry 150 may use the timing control signals to operate synchronously with light drive circuitry 120. For example, front end processing circuitry 150 may synchronize the operation of an analog-to-digital converter and a demultiplexer with the light drive signal based on the timing control signals. In addition, the back end processing circuitry 170 may use the timing control signals to coordinate its operation with front end processing circuitry 150.

Light drive circuitry 120, as discussed above, may be configured to generate a light drive signal that is provided to light source 130 of sensor 102. The light drive signal may, for example, control the intensity of light source 130 and the timing of when light source 130 is turned on and off. When light source 130 is configured to emit two or more wavelengths of light, the light drive signal may be configured to control the operation of each wavelength of light. The light drive signal may comprise a single signal or may comprise multiple signals (e.g., one signal for each wavelength of light). An illustrative light drive signal is shown in FIG. 2A.

In some embodiments, control circuitry 110 and light drive circuitry 120 may generate light drive parameters based on a metric. For example, back end processing circuitry 170 may receive information about received light signals, determine light drive parameters based on that information, and send corresponding information to control circuitry 110.

FIG. 2A shows an illustrative plot of a light drive signal including red light drive pulse 202 and IR light drive pulse 204 in accordance with some embodiments of the present disclosure. Light drive pulses 202 and 204 are illustrated as square waves. As will be described in detail below, these pulses may include shaped waveforms rather than a square wave. The shape of the pulses may be generated by a digital signal generator, digital filters, analog filters, any other suitable equipment, or any combination thereof. For example, light drive pulses 202 and 204 may be generated by light drive circuitry 120 under the control of control circuitry 110. As used herein, drive pulses may refer to the high and low states of a shaped pulse, switching power or other components on and off, high and low output states, high and low values within a continuous modulation, other suitable relatively distinct states, or any combination thereof. The light drive signal may be provided to light source 130, including red light drive pulse 202 and IR light drive pulse 204 to drive red and IR light emitters, respectively, within light source 130. Red light drive pulse 202 may have a higher amplitude than IR light drive pulse 204 because red LEDs may be less efficient than IR LEDs at converting electrical energy into light energy. In some embodiments, the output levels may be equal, may be adjusted for nonlinearity of emitters, may be modulated in any other suitable technique, or any combination thereof. Additionally, red light may be absorbed and scattered more than IR light when passing through perfused tissue.

When the red and IR light sources are driven in this manner they emit pulses of light at their respective wavelengths into the tissue of a subject in order generate physiological signals that physiological monitoring system 100 may process to calculate physiological parameters. It will be understood that the light drive amplitudes of FIG. 2A are merely exemplary and that any suitable amplitudes or combination of amplitudes may be used, and may be based on the light sources, the subject tissue, the determined physiological parameter, modulation techniques, power sources, any other suitable criteria, or any combination thereof.

The light drive signal of FIG. 2A may also include “off” periods 220 between the red and IR light drive pulse. “Off” periods 220 are periods during which no drive current may be applied to, for example, light source 130. “Off” periods 220 may be provided, for example, to prevent overlap of the emitted light, since light source 130 may require time to turn completely on and completely off. The period from time 216 to time 218 may be referred to as a drive cycle, which includes four segments: a red light drive pulse 202, followed by an “off” period 220, followed by an IR light drive pulse 204, and followed by an “off” period 220. After time 218, the drive cycle may be repeated (e.g., as long as a light drive signal is provided to light source 130). It will be understood that the starting point of the drive cycle is merely illustrative and that the drive cycle can start at any location within FIG. 2A, provided the cycle spans two drive pulses and two “off” periods. Thus, each red light drive pulse 202 and each IR drive pulse 204 may be understood to be surrounded by two “off” periods 220. “Off” periods may also be referred to as dark periods, in that the emitters are dark or returning to dark during that period. It will be understood that in some embodiments, light drive schemes other than square pulses may be used, including shaped pulses, sinusoidal modulation, frequency multiplexing, phase multiplexing, any other suitable drive scheme, or any combination thereof.

Referring back to FIG. 1, front end processing circuitry 150 may receive a detection signal from detector 140 and provide one or more processed signals to back end processing circuitry 170. The term “detection signal,” as used herein, may refer to any of the signals generated within front end processing circuitry 150 as it processes the output signal of detector 140. Front end processing circuitry 150 may perform various analog and digital processing of the detector signal. One suitable detector signal that may be received by front end processing circuitry 150 is shown in FIG. 2B.

FIG. 2B shows an illustrative plot of a detector current waveform 214 that may be generated by a sensor in accordance with some embodiments of the present disclosure. The peaks of detector current waveform 214 may represent current signals provided by a detector, such as detector 140 of FIG. 1, when light is being emitted from a light source. The amplitude of detector current waveform 214 may be proportional to the light incident upon the detector. The peaks of detector current waveform 214 may be synchronous with drive pulses driving one or more emitters of a light source, such as light source 130 of FIG. 1. For example, detector current waveform 214 may be generated in response to a light source being driven by the light drive signal of FIG. 2A. Peak 232 may be generated in response to the light source being driven by red light drive pulse 202, and peak 234 may be generated in response to the light source being driven by IR light drive pulse 204. The valleys of detector current waveform 214 may be synchronous with periods of time during which no light is being emitted by the light source, or when the light source is returning to dark. While no light is being emitted by a light source during the valleys, detector current waveform 214 may not fall all of the way to zero.

In some embodiments, one or more samples of the detector current value may be determined at particular points in time. In the illustrated example, the value of the detector current is determined at points 222, 224, 226, 228, and 230. Values may be digitized using analog to digital processing, stored in sample-and-hold analog circuitry, sampled using any other suitable analog technique, sampled using any other suitable digital technique, or any combination thereof. It will be understood that the detector current may be sampled at any suitable rate. For example, one sample per peak and trough may be determined. In another example, samples may be determined at a particular constant rate. It will also be understood that any suitable number of samples may be determined during each respective portion of a drive cycle. For example, more samples may be determined during and “on” portion than an “off” portion.

It will be understood that detector current waveform 214 may be a partially idealized representation of a detector signal, assuming perfect light signal generation, transmission, and detection. It will be understood that an actual detector current may include amplitude fluctuations, frequency deviations, droop, overshoot, undershoot, rise time deviations, fall time deviations, other deviations from the ideal, or any combination thereof.

Referring back to FIG. 1, front end processing circuitry 150, which may receive a detection signal, such as detector current waveform 214, may include analog conditioning 152, demultiplexer 154, digital conditioning 156, analog-to-digital converter (ADC) 158, decimator/interpolator 160, and ambient subtractor 162. Elements of front end processing may be implemented as analog devices, digital devices, and any combination thereof. For example, filtering may be performed using discrete electronic components, integrated electronic components, digital signal processing, any other suitable technique, or any combination thereof.

Analog conditioning 152 may perform any suitable analog conditioning of the detector signal. The conditioning performed may include any type of filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof.

Demultiplexer 154 may operate on the analog or digital form of the detector signal to separate out different components of the signal. For example, detector current waveform 214 of FIG. 2B includes a red component corresponding to peak 232, an IR component corresponding to peak 234, and at least one ambient component, for example corresponding to trough 236. Demultiplexer 154 may operate on detector current waveform 214 of FIG. 2B to generate a red signal, an IR signal, a first ambient signal (e.g., corresponding to trough 236 that occurs immediately after the red component corresponding to peak 232), and a second ambient signal (e.g., corresponding to the trough 238 ambient component that occurs immediately after the IR component corresponding to peak 234). Demultiplexer 154 may operate under the control of control circuitry 110. For example, demultiplexer 154 may use timing control signals from control circuitry 110 to identify and separate out the different components of the detector signal.

Digital conditioning 156 may perform any suitable digital conditioning of the detector signal. Digital conditioning 156 may include any type of digital filtering of the signal (e.g., low pass, high pass, band pass, notch, or any other suitable filtering), amplifying, performing an operation on the signal, performing any other suitable digital conditioning, or any combination thereof.

The conditioned analog signal may be processed by analog-to-digital converter 158, which may convert the conditioned analog signal into a digital signal. Analog-to-digital converter 158 may operate under the control of control circuitry 110. Analog-to-digital converter 158 may use timing control signals from control circuitry 110 to determine when to sample the analog signal. Analog-to-digital converter 158 may be any suitable type of analog-to-digital converter of sufficient resolution to enable a physiological monitor to accurately determine physiological parameters. In some embodiments, analog-to-digital converter 158 may acquire samples at points 222, 224, 226, 228, and 230 of FIG. 2B, as described above.

Decimator/interpolator 160 may decrease the number of samples in the digital detector signal. For example, decimator/interpolator 160 may decrease the number of samples by removing samples from the detector signal or replacing samples with a smaller number of samples. The decimation or interpolation operation may include or be followed by filtering to smooth the output signal.

Ambient subtractor 162 may operate on the digital signal. In some embodiments, ambient subtractor 162 may attenuate dark or ambient contributions to the received signal. A particular embodiment of ambient subtraction using a high pass filter is disclosed in co-pending U.S. application Ser. No. 13/484,808, filed May 31, 2012, entitled “OPTICAL INSTRUMENT WITH AMBIENT LIGHT REMOVAL,” which is hereby incorporated by reference herein in its entirety.

The components of front end processing circuitry 150 are merely illustrative and any suitable components and combinations of components may be used to perform the front end processing operations.

The front end processing circuitry 150 may be configured to take advantage of the full dynamic range of analog-to-digital converter 158. This may be achieved by applying gain to the detection signal by analog conditioning 152 to map the expected range of the detection signal to the full or close to full output range of analog-to-digital converter 158. The output value of analog-to-digital converter 158, as a function of the total analog gain applied to the detection signal, may be given as:

ADC Value=Total Analog Gain×[Ambient Light+LED Light] (1)

Ideally, when ambient light is zero and when the light source is off, the analog-to-digital converter 158 will read just above the minimum input value. When the light source is on, the total analog gain may be set such that the output of analog-to-digital converter 158 may read close to the full scale of analog-to-digital converter 158 without saturating. This may allow the full dynamic range of analog-to-digital converter 158 to be used for representing the detection signal, thereby increasing the resolution of the converted signal. In some embodiments, the total analog gain may be reduced by a small amount so that small changes in the light level incident on the detector do not cause saturation of analog-to-digital converter 158.

However, if the contribution of ambient light is large relative to the contribution of light from a light source, the total analog gain applied to the detection current may need to be reduced to avoid saturating analog-to-digital converter 158. When the analog gain is reduced, the portion of the signal corresponding to the light source may map to a smaller number of analog-to-digital conversion bits. Thus, more ambient light noise in the input of analog-to-digital converter 158 may results in fewer bits of resolution for the portion of the signal from the light source. This may have a detrimental effect on the signal-to-noise ratio of the detection signal. Accordingly, passive or active filtering (e.g., high pass filtering) or signal modification techniques may be employed to reduce the effect of ambient light on the detection signal that is applied to analog-to-digital converter 158, and thereby reduce the contribution of the noise component to the converted digital signal.

Back end processing circuitry 170 may include processor 172 and memory 174. Processor 172 may be adapted to execute software, which may include an operating system and one or more applications, as part of performing the functions described herein. Processor 172 may receive and process physiological signals received from front end processing circuitry 150. For example, processor 172 may determine one or more physiological parameters based on the received physiological signals. Processor 172 may include an assembly of analog or digital electronic components. Processor 172 may calculate physiological information. For example, processor 172 may compute one or more of a pulse rate, respiration rate, blood pressure, or any other suitable physiological parameter. Processor 172 may perform any suitable signal processing of a signal, such as any suitable band-pass filtering, adaptive filtering, closed-loop filtering, any other suitable filtering, and/or any combination thereof. Processor 172 may also receive input signals from additional sources not shown. For example, processor 172 may receive an input signal containing information about treatments provided to the subject from User Interface 180. Additional input signals may be used by processor 172 in any of the calculations or operations it performs in accordance with back end processing circuitry 170 or monitor 104.

Memory 174 may include any suitable computer-readable media capable of storing information that can be interpreted by processor 172. In some embodiments, memory 174 may store calculated values, such as a pulse rate, a blood pressure, a blood oxygen saturation, a fiducial point location or characteristic, an initialization parameter, or any other calculated values, in a memory device for later retrieval. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system. Back end processing circuitry 170 may be communicatively coupled with use interface 180 and communication interface 190.

User interface 180 may include user input 182, display 184, and speaker 186. User interface 180 may include, for example, any suitable device such as one or more medical devices (e.g., a medical monitor that displays various physiological parameters, a medical alarm, or any other suitable medical device that either displays physiological parameters or uses the output of back end processing 170 as an input), one or more display devices (e.g., monitor, personal digital assistant (PDA), mobile phone, tablet computer, smartphone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

User input 182 may include any type of user input device such as a keyboard, a mouse, a touch screen, buttons, switches, a microphone, a joy stick, a touch pad, or any other suitable input device. The inputs received by user input 182 can include information about the subject, such as age, weight, height, diagnosis, medications, treatments, and so forth.

In an embodiment, the subject may be a medical patient and display 184 may exhibit a list of values which may generally apply to the patient, such as, for example, age ranges or medication families, which the user may select using user input 182. Additionally, display 184 may display, for example, an estimate of a subject's blood oxygen saturation generated by monitor 104 (referred to as an “SpO₂” measurement), pulse rate information, respiration rate information, blood pressure, any other parameters, and any combination thereof. Display 184 may include any type of display such as a cathode ray tube display, a flat panel display such a liquid crystal display or plasma display, or any other suitable display device. Speaker 186 within user interface 180 may provide an audible sound that may be used in various embodiments, such as for example, sounding an audible alarm in the event that a patient's physiological parameters are not within a predefined normal range.

Communication interface 190 may enable monitor 104 to exchange information with external devices. Communications interface 190 may include any suitable hardware, software, or both, which may allow monitor 104 to communicate with electronic circuitry, a device, a network, a server or other workstations, a display, or any combination thereof. Communications interface 190 may include one or more receivers, transmitters, transceivers, antennas, plug-in connectors, ports, communications buses, communications protocols, device identification protocols, any other suitable hardware or software, or any combination thereof. Communications interface 190 may be configured to allow wired communication (e.g., using USB, RS-232, Ethernet, or other standards), wireless communication (e.g., using WiFi, IR, WiMax, BLUETOOTH, USB, or other standards), or both. For example, communications interface 190 may be configured using a universal serial bus (USB) protocol (e.g., USB 2.0, USB 3.0), and may be configured to couple to other devices (e.g., remote memory devices storing templates) using a four-pin USB standard Type-A connector (e.g., plug and/or socket) and cable. In some embodiments, communications interface 190 may include an internal bus such as, for example, one or more slots for insertion of expansion cards.

It will be understood that the components of physiological monitoring system 100 that are shown and described as separate components are shown and described as such for illustrative purposes only. In some embodiments the functionality of some of the components may be combined in a single component. For example, the functionality of front end processing circuitry 150 and back end processing circuitry 170 may be combined in a single processor system. Additionally, in some embodiments the functionality of some of the components of monitor 104 shown and described herein may be divided over multiple components. For example, some or all of the functionality of control circuitry 110 may be performed in front end processing circuitry 150, in back end processing circuitry 170, or both. In other embodiments, the functionality of one or more of the components may be performed in a different order or may not be required. In an embodiment, all of the components of physiological monitoring system 100 can be realized in processor circuitry.

FIG. 3 is a perspective view of an embodiment of a physiological monitoring system 310 in accordance with some embodiments of the present disclosure. In some embodiments, one or more components of physiological monitoring system 310 may include one or more components of physiological monitoring system 100 of FIG. 1. Physiological monitoring system 310 may include sensor unit 312 and monitor 314. In some embodiments, sensor unit 312 may be part of an oximeter. Sensor unit 312 may include one or more light sources 316 for emitting light at one or more wavelengths into a subject's tissue. One or more detector 318 may also be provided in sensor unit 312 for detecting the light that is reflected by or has traveled through the subject's tissue. Any suitable configuration of light source 316 and detector 318 may be used. In an embodiment, sensor unit 312 may include multiple light sources and detectors, which may be spaced apart. Physiological monitoring system 310 may also include one or more additional sensor units (not shown) that may, for example, take the form of any of the embodiments described herein with reference to sensor unit 312. An additional sensor unit may be the same type of sensor unit as sensor unit 312, or a different sensor unit type than sensor unit 312 (e.g., a photoacoustic sensor). Multiple sensor units may be capable of being positioned at two different locations on a subject's body.

In some embodiments, sensor unit 312 may be connected to monitor 314 as shown. Sensor unit 312 may be powered by an internal power source, e.g., a battery (not shown). Sensor unit 312 may draw power from monitor 314. In another embodiment, the sensor may be wirelessly connected to monitor 314 (not shown). Monitor 314 may be configured to calculate physiological parameters based at least in part on data relating to light emission and acoustic detection received from one or more sensor units such as sensor unit 312. For example, monitor 314 may be configured to determine pulse rate, blood pressure, blood oxygen saturation (e.g., arterial, venous, or both), hemoglobin concentration (e.g., oxygenated, deoxygenated, and/or total), any other suitable physiological parameters, or any combination thereof. In some embodiments, calculations may be performed on the sensor units or an intermediate device and the result of the calculations may be passed to monitor 314. Further, monitor 314 may include display 320 configured to display the physiological parameters or other information about the system. In the embodiment shown, monitor 314 may also include a speaker 322 to provide an audible sound that may be used in various other embodiments, such as for example, sounding an audible alarm in the event that a subject's physiological parameters are not within a predefined normal range. In some embodiments, physiological monitoring system 310 may include a stand-alone monitor in communication with the monitor 314 via a cable or a wireless network link. In some embodiments, monitor 314 may be implemented as display 184 of FIG. 1.

In some embodiments, sensor unit 312 may be communicatively coupled to monitor 314 via a cable 324. Cable 324 may include electronic conductors (e.g., wires for transmitting electronic signals from detector 318), optical fibers (e.g., multi-mode or single-mode fibers for transmitting emitted light from light source 316), any other suitable components, any suitable insulation or sheathing, or any combination thereof. Cable 324 may connect to monitor 314 at port 336. In some embodiments, a wireless transmission device (not shown) or the like may be used instead of or in addition to cable 324. Monitor 314 may include a sensor interface configured to receive physiological signals from sensor unit 312, provide signals and power to sensor unit 312, or otherwise communicate with sensor unit 312. The sensor interface may include any suitable hardware, software, or both, which may be allow communication between monitor 314 and sensor unit 312.

In some embodiments, physiological monitoring system 310 may include calibration device 380. Calibration device 380, which may be powered by monitor 314, a battery, or by a conventional power source such as a wall outlet, may include any suitable calibration device. Calibration device 380 may be communicatively coupled to monitor 314 via communicative coupling 382, and/or may communicate wirelessly (not shown). In some embodiments, calibration device 380 is completely integrated within monitor 314. In some embodiments, calibration device 380 may include a manual input device (not shown) used by an operator to manually input reference signal measurements obtained from some other source (e.g., an external invasive or non-invasive physiological measurement system).

In the illustrated embodiment, physiological monitoring system 310 includes a multi-parameter physiological monitor 326. The monitor 326 may include a cathode ray tube display, a flat panel display (as shown) such as a liquid crystal display (LCD) or a plasma display, or may include any other type of monitor now known or later developed. Multi-parameter physiological monitor 326 may be configured to calculate physiological parameters and to provide a display 328 for information from monitor 314 and from other medical monitoring devices or systems (not shown). For example, multi-parameter physiological monitor 326 may be configured to display an estimate of a subject's blood oxygen saturation and hemoglobin concentration generated by monitor 314. Multi-parameter physiological monitor 326 may include a speaker 330.

Monitor 314 may be communicatively coupled to multi-parameter physiological monitor 326 via a cable 332 or 334 that is coupled to a sensor input port or a digital communications port, respectively and/or may communicate wirelessly (not shown). In addition, monitor 314 and/or multi-parameter physiological monitor 326 may be coupled to a network to enable the sharing of information with servers or other workstations (not shown). Monitor 314 may be powered by a battery (not shown) or by a conventional power source such as a wall outlet.

In some embodiments, all or some of monitor 314 and multi-parameter physiological monitor 326 may be referred to collectively as processing equipment.

In some embodiments, any of the processing components and/or circuits, or portions thereof, of FIGS. 1 and 3 may be referred to collectively as processing equipment. For example, processing equipment may be configured to amplify, filter, sample and digitize an input signal from sensor 102 (e.g., using an analog-to-digital converter), and calculate physiological information from the digitized signal. Processing equipment may be configured to generate light drive signals, amplify, filter, sample and digitize detector signals, and calculate physiological information from the digitized signal. In some embodiments, all or some of the components of the processing equipment may be referred to as a processing module.

FIG. 4 is a block diagram of optical monitoring system 400 in accordance with some embodiments of the present disclosure. System 400 includes light drive module 402, transmission module 404, LED module 406, tissue 408, detector module 410, receiving module 412, equalizer module 414, and processing module 416. In some embodiments, channel 418 includes transmission module 404, LED module 406, detector module 410, and receiving module 412, as indicated by the dotted line. It will be understood that this particular border of the channel is merely exemplary and that any suitable elements or modules may be included or excluded from the channel. Equalizer module 414 may be configured to mitigate undesired signal features associated with components within channel 418 from the signal passed to processing module 416. In some embodiments, equalizer may mitigate signal features associated with channel elements while features associated with light drive module 402 and tissue 408 preferably remain substantially or wholly unchanged.

In some embodiments, system 400 includes some elements described in physiological monitoring system 100 of FIG. 1. Light drive module 402 may include any suitable hardware, software, or combination thereof, used to generate a light drive signal. For example, a light drive module 402 may include some or all of light drive circuitry 120 of FIG. 1. In some embodiments, light drive module 402 may generate a signal such as the light drive signal of FIG. 2A.

In some embodiments, transmission module 404 includes any suitable connections between the generation of a signal by light drive module 402 and LED module 406. For example, transmission may include connectors, metallic conductors, wireless communications, any other suitable technique for transmitting a signal to LED module 406, and any combination thereof. In some embodiments, transmission module 404 may include amplifiers, filters, any other suitable conditioning techniques, or any combination thereof. In some embodiments, transmission module 404 may introduce signal distortions, noise, and other undesired signal features. Undesired features may be caused by non-ideal signal conduction, RF interference, parasitic capacitances, any other suitable source, and any combination thereof.

LED module 406 may include some or all of light source 130 of FIG. 1. In some embodiments, LED module 406 receives an electrical signal from transmission module 404 and generates an optical signal based on the received signal. For example, LED module 406 may include one or more LEDs. In some embodiments, LED module 406 may introduce signal distortions and other undesired signal features due to, for example, non-ideality of the electrical to optical conversion of LEDs, light source turn-on and turn-off rates, temperature-dependent parameters of the light source, any other suitable source, or any combination thereof.

Tissue 408 may include any suitable test subject tissue. For example, in a finger clip oximeter sensor, tissue 408 may include the finger of a test subject such as a patient. Tissue 408 may also include a calibration or testing sample such as a block of plastic. In some embodiments, tissue 408 may be omitted, for example in a calibration measurement. In some embodiments, tissue 408 may be opaque to the light, for example, in a calibration measurement. In some embodiments, as shown, tissue 408 is not included in channel 418.

Detector module 410 may include some or all of detector 140 of FIG. 1. For example, in some embodiments, detector module 410 receives a portion of the light emitted from LED module 406 and converts the optical signal to an electrical signal.

Receiving module 412 includes any suitable communication and processing components that communicate the signal output by detector module 410 to equalizer module 414. In some embodiments, receiving module 412 may include signal transmission elements such as those described for transmission module 404. In some embodiments, receiving module 412 may include a high pass filter or other suitable filter configured to attenuate ambient light or other light not emitted by LED module 406 from the signal. In some embodiments, receiving module 412 may include some or all of front end processing circuitry 150 of FIG. 1. For example, receiving may include analog-to-digital converter 158 of FIG. 1. It will be understood that in some embodiments, receiving module 412 may include no elements of front end processing 150. In some embodiments, receiving module 404 may include all elements of the system prior to equalizer module 414, and thus any elements between the detector and equalizer module 414 may be included in channel 418.

Equalizer module 414 includes any suitable hardware, software, or combination thereof, to perform equalization of the received signal. In some embodiments, equalizer module 414 mitigates channel effects associated with channel 418. In some embodiments, equalizer module 414 may include one or more time or frequency domain elements where a particular amount of gain is applied to each respective element. In some embodiments, equalizer module 414 may include discrete processing, continuous processing, or any combination thereof.

In some embodiments, equalizer module 414 performs equalization in the time domain, for example by using a filter such as an FIR filter. In some embodiments, time domain equalization includes a finite impulse response (FIR) filter. An FIR filter includes an impulse response of finite duration, such that in discrete time filtering, a series of filter coefficients is applied to the current sample and a number of adjacent samples. In an example, the output of a discrete time FIR may be a weighted sum of the current and a particular number of previous samples, where the weighting coefficients are the filter coefficients. FIR filters may include discrete time filtering, continuous time filtering, or any combination thereof. FIR filters may be implemented in digital processing, analog processing, hardware, software, any other suitable implementation, or any combination thereof. In some embodiments the system may include an infinite impulse response (IIR), a least mean squares (LMS) filter, a root mean square filter (RMS), an adaptive filter, any other suitable type of filter, or any combination thereof. An adaptive filter may, for example, adapt to changes that occur over time, but at a slower rate than physiological changes. In some embodiments, more than one filter of any suitable type may be used.

In some embodiments, a channel response may be approximated as a filter function h in the time domain. For example, where h is a finite impulse response (FIR) filter. Thus, if the input to the channel is x(t) and the output of the channel is y(t), the output y(t) includes the convolution of x(t) and h:

y(t)=x(t)*(h) (2)

where * includes the convolution function. In an embodiment where the signal passes through a channel and also passes through a test subject, the output from the system may include:

y(t)=x(t)*(h+S) (3)

where S includes the response of the test subject. In some embodiments, the signal may be equalized to remove the response h so that physiological information corresponding to response S may be determined. By the distributive property of convolution:

y(t)=(x(t)*h)+(x(t)*S) (4)

The system may apply a channel equalizer to remove the channel response h:

y_{equalized}(t)=y(t)−(x(t)*(h))=x(t)*S (5)

Such that the equalized signal y_{equalized}(t) represents primarily the response of the test subject with the channel response removed.

In some embodiments, equalizer module 414 optionally receives a signal from light drive module 402, as indicated by the dotted line. The received signal may include the signal sent to transmission module 404, information about the light drive signal, any other suitable information, or any combination thereof. In some embodiments, equalizer module 414 may use the received information as a reference signal in equalizing the signal received from receiving module 412. For example, where equalizer module 414 includes an FIR filter, the system may perform a convolution of the light drive signal and the filter coefficients, and then subtract the result of the convolution from the output of the signal from receiving module 412. In another example, the system may use the reference signal from light drive module 402 to calibrate, adjust, or refine filter coefficients or other parameters of equalizer module 414 in a calibration step and/or during operation.

In some embodiments, equalization may be performed in the frequency domain, for example by using a transform of a time domain signal such as a Fast Fourier Transform (FFT), followed by a complex multiplication of one or more FFT frequency bins. In some embodiments, convolution functions in the time domain may be replaced with multiplication functions in the frequency domain. For example, where each frequency bin output from a transform into the frequency domain is multiplied by a complex number, each frequency will include an amplitude scaling and a phase shift (i.e., a complex rotation) to mitigate channel effects. Coefficients used in the complex multiplication in the frequency domain may be determined by any of the calibration and modeling techniques described herein. For example, where the signal entering a channel is x(t) and output is y(t), frequency domain signal Y(f) may be determined using a fast Fourier transform as FFT (y(t)), and the amplitude and phase shift at each frequency f may be measured. A frequency domain equalizer function H(f) may be calculated such that Y(f)*H(f) mitigates the frequency and phase effects (i.e., scaling and rotation) associated with the channel response.

It will be understood that equalization may include time domain equalization, frequency domain equalization, or any combination thereof. In some embodiments, the output of the equalizer may include either time domain (e.g., time domain signals processed in the frequency domain may be converted back to the time domain using an inverse FFT), frequency domain signals, or any combination thereof. In some embodiments, filtering and/or processing of the received signals may be interleaved with one or more equalizer stages in any suitable order. In some embodiments, light source drive pulses may be shaped to pre-compensate for channel effects. Pre-shaping is disclosed in co-pending patent U.S. application Ser. No. ______, filed ______, entitled “METHODS AND SYSTEMS FOR SHAPING DRIVE PULSES IN A MEDICAL DEVICE” (Attorney Docket Number H-RM-02723 (CVDN-062), which is hereby incorporated by reference in its entirety.

Processing module 416 may include any suitable elements of front end processing 150 of FIG. 1, back end processing 170 of FIG. 1, any other suitable processing, or any combination thereof. In some embodiments, processing module 416 receives an equalized signal from equalizer module 414. In some embodiments, the received equalized signal includes signal features associated with tissue 408 while signal features associated with channel 418 have been mitigated.

FIG. 5 shows illustrative signal chain 500 that equalizes a signal in accordance with some embodiments of the present disclosure. In some embodiments, signal chain 500 illustrates various elements of system 400 of FIG. 4.

In square input 502, an exemplary light drive signal is depicted as an ideal square waveform. In an example, the square light drive signal is the light drive signal generated by light drive module 402 of FIG. 4. It will be understood that the input signal may be any suitable signal. For example, signals may include sinusoidal waves, shaped pulses, frequency division signals, any suitable time division multiplexing, any suitable frequency division multiplexing, or any combination thereof.

The square waveform from square input 502 passes through channel 504. In some embodiments, channel 504 includes elements described for channel 418 of FIG. 4. In some embodiments, channel 504 represents the path of the signal between the generation of the light drive signal and the processing of the received, digitized signal. The channel may include, for example, LEDs or other light emitters, optical filters, lenses, housings, analog filter components (e.g., capacitors, inductors, resistors, and amplifiers), digital filter components, photodetectors, analog-to-digital converters, interconnects, any other suitable components, or any combination thereof. In an example, channel 504 may include any combination of components included in light drive circuitry 120 of FIG. 1, sensor 102 of FIG. 1, and front end processing circuitry 150 of FIG. 1. For example, channel 504 may include the portions of the system where analog signals are transmitted or processed.

In some embodiments, channel 504 distorts and/or otherwise alters square input 502, resulting in distorted output 506. Distorted output includes a square wave with undesired signal features. For example, the square wave may include droop (as shown), overshoot, ringing, any other suitable feature, or any combination thereof. For example, channel 504 may include ambient subtractor 162 of FIG. 1, which may introduce droop into a square wave signal. The portion of the channel response attributable to high pass filtering for ambient light subtraction can be mitigated, as described for example in co-pending U.S. application Ser. No. 13/484,808, filed May 31, 2012, entitled “OPTICAL INSTRUMENT WITH AMBIENT LIGHT REMOVAL,” which is hereby incorporated by reference herein in its entirety. For example, where ambient light subtraction is implemented using a high pass filter, the high pass filter attenuates the low frequency components of a detector signal. In some embodiments, a high pass filter introduces undesired droop in a square wave signal. Droop is a signal feature where the voltage level of a square wave during a pulse decreases over time rather than remaining constant. Overshoot is observed when the rising edge of a pulse initially exceeds the intended output voltage before settling to the intended voltage. Undershoot is observed when the rising edge of a pulse fails to initially reach the intended output voltage before settling to the intended voltage. Ringing is observed when a voltage output oscillates above and below an intended value. Rise and fall time distortions are observed when the system reacts slowly to the change of a rising or falling edge of a pulse. It will be understood that the aforementioned signal features are merely exemplary and that any suitable signal features may be introduced to a signal by the channel.

In some embodiments, the undesired signal features are relatively constant from peak to peak. In some embodiments, equalizer 508 receives distorted output 506 and outputs square output 510. In some embodiments, square output 510 represents the input signal with the undesired signal features mitigated. In some embodiments, where the signal features are relatively constant and repeating, equalizer 508 may mitigate those known and/or predictable features. In some embodiments, features may be mitigated by introducing an inverse of the undesired signal features, such that the undesired features are mitigated and the desired features remain. For example, a finite impulse response filter equalizer may apply the inverse of the channel response to the signal. In another example, described in further detail below in FIG. 7, predetermined equalizer gains are applied at various times to a signal generated using a periodic light drive signal to attenuate droop.

In the illustrated example of signal chain 500, the signal does not pass through a test subject such as tissue 408 of FIG. 4. In some embodiments, the output of a signal passing through both a channel and a test subject would include both desired and undesired signal features. An equalizer such as equalizer 508 may mitigate only the undesired features, such as those associated with the channel but not with the test subject. In the illustrated example, where there is no test subject, the equalizer mitigates the signal features of the channel and returns the original square shape input. In an embodiment where a test subject is present, the output of the equalizer may substantially include the desired signal features associated with the test subject.

FIG. 6 shows illustrative signal chain 600 including a test subject in accordance with some embodiments of the present disclosure. In some embodiments, signal chain 600 illustrates the signals illustrated in signal chain 500 when a test subject is included. In some embodiments, signal chain 600 illustrates various elements of system 400 of FIG. 4.

Signal chain 600 includes square input 602. In some embodiments, square input is configured as described for square input 502 of FIG. 5. As described above, it will be understood that the input signal may be any suitable signal. For example, signals may include sinusoidal waves, shaped pulses, frequency division signals, any suitable time division multiplexing, any suitable frequency division multiplexing, or any combination thereof.

The square waveform from square input 602 passes through channel 604. In some embodiments, channel 604 includes elements described for channel 418 of FIG. 4. In some embodiments, channel 604 is configured as described for channel 504 of FIG. 5.

After interacting with a portion of channel 604, the signal interacts with tissue 612. Tissue 612 may correspond to tissue 408 of FIG. 4. For example, tissue 612 may include a test subject such as a fingertip, earlobe, or other sensing location, a calibration sample, a calibration block of polytetrafluoroethylene, any other suitable material or combination of materials, or any combination thereof. In some embodiments, tissue 612 includes physiological tissue.

In some embodiments, a signal interacts with the first part of channel 604, followed by interacting with tissue 612, followed by interacting with a second part of channel 604. In some embodiments, this corresponds to the signal path illustrated for channel 418 of FIG. 4 and tissue 408 of FIG. 4. It will be understood that the particular sequence illustrated in signal chain 600 and in FIG. 4 is merely exemplary and that the tissue and channel elements may be arranged and/or interspersed in any suitable order.

Output 606 includes the signal as it is output from the channel before reaching an equalizer. For example, output 606 may correspond to the output of receiving module 412 of FIG. 4. In some embodiments, output 606 includes the input signal combined with both physiological attenuations and other effects, and channel attenuations and other effects. For example, as illustrated, output 606 includes a square wave that includes peak height attenuation, phase shifts, droop and rise distortions, and other desirable and undesirable features. For example, the peak height attenuations may primarily correspond to physiological information while the phase shifts correspond to channel effects, however the phase shifts (and other channel effects) may impact the physiological information by also altering peak heights.

Equalizer 608 receives output 606 and generates equalized output 610. Equalizer 608 may correspond to equalizer module 414 of FIG. 4, equalizer 508 of FIG. 8, any other suitable equalizer, or any combination thereof. For example, equalizer 608 may include an FIR filter that equalizes the samples in order to remove channel effects. As illustrated, equalized output 610 shows that the attenuation of the peak heights corresponding to physiological information is retained in the equalized signal while the phase shifts and other undesirable channel responses have been removed. For example, the peaks heights of the square waves may be used to determine the attenuation of red and IR light pulses, from which a blood oxygen saturation value may be determined.

FIG. 7 shows illustrative signals including time domain equalization in accordance with some embodiments of the present disclosure. It will be understood that FIG. 7 illustrates a particular exemplary type of time domain equalization that may be used additionally or alternatively to an FIR filter. The time domain equalizer of FIG. 7 receives periodic samples and applies a periodic set of gains to each respective sample.

FIG. 7 includes distorted signal 702 and equalized signal 710. In an example, a high pass filter in the signal channel may introduce a droop in a square wave, as shown. Equalized signal 710 is generated by equalizer 708. Distorted signal 702 may correspond to distorted output 506 of FIG. 5. Equalizer 708 may correspond to equalizer 508 of FIG. 5 and/or equalizer module 414 of FIG. 4. Equalized signal 710 may correspond to square output 510 of FIG. 5 and/or the output of equalizer module 414 of FIG. 4. In some embodiments, one or more time points of signal 702 are sampled, for example, as illustrated for detector current waveform 214 of FIG. 2B. For example, distorted signal 702 may be sampled at time points 704, 706, and at the other circles illustrated on distorted signal 702. It will be understood that in some embodiments, the entirety of distorted signal 702 may not be received by equalizer 708 and that only the sampled data points may be received. In another example, equalizer 708 may receive distorted signal 702 as an analog signal and may both sample and equalize.

In some embodiments, equalizer 708 includes one or more elements. For example, equalizer may include element 716 that receives the level of distorted signal 702 at time point 704, element 718 that receives the level of distorted signal 702 at time point 706, and additional elements, as depicted, that each receive a level of distorted signal 702 at a different point in time. In some embodiments, equalizer may apply a particular amount of gain or other suitable signal adjustment in each respective element to generate an output. As shown, the element 716 generates the output level of equalized signal 710 at time point 712. The element 718 generates the output level of equalized signal 710 at time point 714.

In some embodiments, the signal adjustments applied by each element of equalizer 708 are configured to mitigate some or all of the undesired signal features of distorted signal 702. For example, undesired signal features may be associated with channel effects, as described above. In an example, particular amounts of gain may be applied to each input of equalizer 708 such that undesired features such as the illustrated droop are mitigated in the output. In some embodiments, the undesired signal features introduced by the channel (e.g., for each pulse) are relatively constant. Accordingly, the equalizer gain levels may be predetermined in a calibration and/or modeling step. These predetermined gain levels may be applied to a signal including test subject features, such that channel signal features are mitigated. Thus, the output of the equalizer may substantially include only the effects of the test subject on the light drive signal.

Gains associated with an equalizer such as equalizer 708 may be determined in a calibration step, in device design, based on modeling and/or testing of components in the signal channel, based on identified components in a particular installation or use, based on any other suitable information, or any combination thereof. For example, the equalizer gains may be adjusted while a test subject is not present until the output of the equalizer is as desired, and subsequently a test subject may be introduced. In another example, the elements of the channel may be modeled computationally to determine their impact on a signal, and gains may be determined based on those computations. In another example, particular system elements of the channel, such as different cables or light sources, may be associated with particular impacts on the signal, and equalizer gains may be set based on the presence or absence of those particular system elements. In some embodiments, the system may automatically detect a cable assembly (e.g., by interrogating the cable assembly or by any other suitable technique). In another example, calibration includes training signals. Training signals may include light drive signals that are used to set equalizer gains. For example, training signals may include typical light drive signals, signals that include a relatively wide dynamic range of inputs, any other suitable signal, or any combination thereof.

It will be understood that the above-described implementation of time domain equalization is merely exemplary. For example, in some embodiments the system may perform equalization in another domain such as the frequency domain. It will also be understood that the particular number of equalization channels is merely exemplary. It will also be understood that the system may apply equalization to both the high and low portions of a square wave, or to any suitable portions of any suitable light drive signal. For example, the signal may be a sinusoid, and the system may apply periodic time or frequency domain equalization.

FIG. 8 shows an illustrative flow diagram 800 including steps for determining a physiological parameter in accordance with some embodiments of the present disclosure.

In step 802, the system receives a light signal. A light signal may be received from a detector such as detector 140 of FIG. 1, detector module 410 of FIG. 4, any other receiver, or any combination thereof. In some embodiments, an optical light signal is received by an optical-electrical photodetector, which generates an electrical signal corresponding to the optical signal. In some embodiments, the system may receive a light signal that corresponds to a light signal detected in any suitable manner. In some embodiments, detector current waveform 214 of FIG. 2B may correspond to a received light signal.

In some embodiments, the received light signal corresponds to an emitted light signal. In some embodiments, the received light signal corresponds to an emitted light signal that has been partially attenuated by a test subject, for example, tissue 408 of FIG. 4. In some embodiments, the emitted light signal may emitted from a light source such as light source 130 of FIG. 1, LED module 406 of FIG. 4, any other suitable light source, or any combination thereof. The light source may include one or more light emitting diodes, laser light sources, wide spectrum light sources, fluorescent light sources, light filters, any other suitable source, or any combination thereof. For example, the light source may include a red LED and an infrared LED, and the received light signal may include a pattern of red and IR led light, as shown in the illustrative light drive signal of FIG. 2A. The pattern may include repeating portions where light is emitted during a first portion and not emitted during a second portion.

In step 804, the system determines multiple samples of the light signal. In some embodiments, the system may determine a voltage, current, power, or other suitable level of a signal at multiple points in time. For example, samples may be determined as illustrated for detector current waveform 214 of FIG. 2B. In another example, samples may be determined as described for distorted signal 702 of FIG. 7. In an example, the system may determine multiple samples of an analog signal, where each sample is a voltage level at a particular time. When the light signal includes a pattern of repeating portions, multiple samples may be determined for one or more of the repeating portions. Sampling rate may be determined based on the frequency of a periodic light drive signal, based on a system design parameter, based on a noise level, based on any other suitable parameter, or any combination thereof. In another example, the signal may be converted to the frequency domain and samples may be determined according to multiple frequency bands at particular points in time. In another example, time domain samples may be used to determine a frequency domain signal. It will be understood that the aforementioned determination of samples is merely exemplary and that the system may determine any suitable number of samples using any suitable technique.

In step 806, the system equalizes the samples determined in step 804. Equalization may include time domain equalization, frequency domain equalization, a finite impulse response filter, an infinite impulse response filter, adaptive filters including LMS implementation or any other suitable equalization technique, or any combination thereof. For example, time domain equalization may include applying a periodic set of gains to samples in the time domain. In another example, frequency domain equalization may include applying a set of complex multiplications to some or all frequency bands. In another example, finite impulse response equalization may include applying a finite impulse response filter to the samples. For example, a finite impulse response filter may apply the inverse of the channel frequency response to the samples of the received light signal.

In some embodiments, gains are determined based on channel response, such as the frequency response of channel 418 of FIG. 4. The channel response may include features such as droop, rise time, fall time, overshoot, undershoot, high amplitude, low amplitude, ringing, any other suitable feature, or any combination thereof. In some embodiments, gains may be determined to reduce and/or eliminate these features.

In some embodiments, equalizing the samples includes using a gain. Gains may be fixed, variable, or any combination thereof. Different gains and types of gains may be applied to each sample determined in step 804. For example, where the detected light signal is substantially periodic (e.g., in a pattern of repeating portions), a periodic set of gains may be applied such that each respective sample of the signal period has the same gain applied to it. In an example, a high pass filter element in a channel results in a channel response to a square wave input that includes droop. Increasing amounts of gain may be applied as the period of the signal progresses to counteract the droop. For example, gain may be increased for each subsequent sample in a repeating portion of the detected light signal.

Gains may be determined based on modeling of components in the channel, based on training signals, based on calibration, based on system design, based on user input, based on any other suitable information, or any combination thereof. Modeling of components in the channel may include computational modeling of the frequency response of elements of the channel, for example filters and amplifiers. Elements may be modeled collectively, individually, or any combination thereof. In some embodiments, a modular technique may be used, where the system modifies gains based on the particular set of channel elements included. For example, a known channel response may be associated with a particular fingertip sensor connected to the system, while another known channel response may be associated with a forehead sensor. Modeling may occur at startup and/or initialization of the system, in system design, during system use, at any other suitable time, or any combination thereof. Training signals and calibration may be used additionally or alternatively to channel modeling. For example, a training signal may be passed through the channel when a test subject is not included, and the frequency response of the channel to the training signal may be used to determine gains. For example, the inverse of the channel response to a training signal may be set as the response of a finite impulse response filter. The system may use calibration and training signals at system startup and/or initialization, in a system design stage, during use at any suitable interval, when the system determines calibration is needed due to a change in confidence, noise, and/or other parameters, when changing system components such as probes and/or light sources, at any other suitable time, or any combination thereof.

In step 808, the system determines a physiological parameter of the subject based on the equalized samples of step 806. In some embodiments, the system determines pulse rate, respiration rate, respiration effort, blood oxygen saturation, blood pressure, any other suitable physiological parameter, or any combination thereof.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed:

1. A method for determining an equalized physiological signal, the method comprising:
receiving, using processing equipment, a light signal representing light attenuated by a subject, wherein the light signal comprises first and second repeating portions, and wherein the light signal includes an undesired channel response of a channel; and
equalizing, using processing equipment, the light signal to mitigate the undesired channel response.

2. The method of claim 1, wherein more light is emitted during the first repeating portion than during the second repeating portion.

3. The method of claim 1, wherein equalizing the signal comprises equalizing using a finite impulse response (FIR) filter.

4. The method of claim 1, wherein equalizing the light signal comprises equalizing using a time domain equalizer.

5. The method of claim 1, wherein the undesired channel response comprises a phase shift, a frequency dependent phase shift, a droop, a rise time, a fall time, an overshoot, an undershoot, a ringing, or any combination thereof.

6. The method of claim 1, wherein equalizing the light signal comprises equalizing based on a model of components in the channel.

7. The method of claim 1, wherein equalizing the light signal comprises equalizing based on training.

8. The method of claim 1, wherein the undesired channel response comprises a droop generated based on a high pass filter effect of the channel.

9. The method of claim 1, wherein the undesired channel response comprises a phase shift.

10. The method of claim 1, further comprising determining, using the processing equipment, a physiological parameter of the subject based on the equalized light signal.

11. The method of claim 10, wherein determining a physiological parameter comprises determining a parameter selected from the group consisting of blood oxygen saturation, blood pressure, heart rate, respiration rate, respiration effort, and any combination thereof.

12. A system for determining an equalized physiological signal, comprising:
processing equipment configured to:
receive a light signal representing light attenuated by a subject, wherein the light signal comprises first and second repeating portions, and wherein the light signal includes an undesired channel response of a channel; and
equalize the light signal to mitigate the undesired channel response.

13. The system of claim 12, wherein more light is emitted during the first repeating portion than during the second repeating portion.

14. The system of claim 12, wherein the processing equipment is configured to use a finite impulse response (FIR) filter to equalize the light signal.

15. The system of claim 12, wherein the processing equipment is configured to use a time domain equalizer to equalize the light signal.

16. The system of claim 12, wherein the undesired channel response comprises a phase shift, a frequency dependent phase shift, a droop, a rise time, a fall time, an overshoot, an undershoot, a ringing, or any combination thereof.

17. The system of claim 12, wherein the processing equipment is further configured to equalize the light signal based on a model of components in the channel.

18. The system of claim 12, wherein the processing equipment is further configured to equalize the light signal based on training.

19. The system of claim 12, wherein the undesired channel response comprises a droop generated based on a high pass filter effect of the channel.

20. The system of claim 12, wherein the undesired channel response comprises a phase shift.

21. The system of claim 12, wherein the processing equipment is further configured to determine a physiological parameter of the subject based on the equalized light signal.

22. The system of claim 21, wherein the physiological parameter is selected from the group consisting of blood oxygen saturation, blood pressure, heart rate, respiration rate, respiration effort, and any combination thereof.
